(19) United States
(12) Patent Application Publication
(10) Pub. No.: US 2010/0331577 A1
(43) Pub. Date: Dec. 30, 2010

(54) Method for radio-labeling serotonin transporter ligand, 123I-IADM

(75) Inventors: Chia-Chieh Chen, Longtan Shiang (TW); Shih-Ying Lee, Longtan Shiang (TW); Hung-Chun Kao, Longtan Shiang (TW); Kang-Wei Chang, Longtan Shiang (TW); Li-Chung Hwang, Longtan Shiang (TW)

Correspondence Address: Jackson Intellectual Property Group PLLC, 106 Starvale Lane, Shipman, VA, 22971, US

(73) Assignee: ATOMIC ENERGY COUNCIL-INSTITUTE OF NUCLEAR ENERGY RESEARCH, Taoyuan (TW)

(21) Appl. No.: 12/081,947

(22) Filed: Apr. 23, 2008

(51) Int. Cl. C07C 211/29

(52) U.S. Cl. 564/341

(57) ABSTRACT

There is disclosed a method for using ¹²³I to radiolabel SnADAM become an serotonin transporter radiotracer(¹²³I -ADAM). At first, there is provided SnADAM solution via mixing SnADAM with ethanol. The SnADAM solution is shaken and further mixed with thin KI solution. The SnADAM solution is mixed with ¹²³I-NH₄I solution and H₂O₂ solution. The SnADAM-¹²³I-NH₄I—H₂O₂ mixture is kept still. Later, the SnADAM solution is mixed with NaHSO₃ solution, and the mixture is shaken and further mixed with buffer solution of saturated Na₂HPO₄. The SnADAM solution is filled in an Accubond C8 column. The Accubond C8 column is washed with sterile water for injection to isolate non-reacting ¹²³I ions. The Accubond C8 column is washed with ethanol, thus providing ¹²³I-ADAM. The ¹²³I-ADAM is blended in normal saline mixture. Millipore Millex GV is used to filter impurities and bacteria from the ¹²³I-ADAM solution.

### FIELD OF THE INVENTION

The present invention relates to a method for radio-labeling a serotonin transporter ligand, ¹²³I-ADAM.

### DESCRIPTION OF THE RELATED ARTS

In 1999, Kung et al. announced a serotonin transporter ligand, ¹²⁵I-IDAM, which is lipophilic and is suitable for penetrating blood-brain-barrier. Two minutes after injection, the absorption of ¹²⁵I-IDAM by a brain is 2.44% which is good for the imagining of the serotonin transporter. However, the specific binding affinity of ¹²⁵I-IDAM is 1.75 so that the image of the serotonin transporter is not clear enough.

In 2000, Kung et al. announced a derivative of ¹²⁵I-IDAM, i.e., ¹²⁵I-ODAM. The specific binding affinity of ¹²⁵I-ODAM with SERT is good as proven in an in vitro specific binding assay. However, an image obtained with the use of ¹²⁵I-ODAM is less clear than with the use of ¹²⁵I-IDAM.

Later, Kung et al announced another derivative from ¹²⁵I-IDAM, i.e., ¹²⁵I-ADAM, which is lipophilic like ¹²⁵I-IDAM. Although the absorption of ¹²⁵I-ADAM by the brain is less than ¹²⁵I-IDAM, the affinity of ¹²⁵I-ADAM with the serotonin transporter is much better than that of ¹²⁵I-IDAM. Therefore, the specific binding affinity of ¹²⁵I-ADAM is much higher than ¹²⁵I-IDAM so that the image is clearer and diagnosis based on the image is the more reliable.

In 2003, Halldin et al. announced [³H]MADAM and [³C]DADAM for use in positron emission tomography. [³H]MADAM and [³C]DADAM are derivatives of ¹²⁵I-IDAM, and their affinity with the serotonin transporter is high.

The present invention is therefore intended to obviate or at least alleviate the problems encountered in prior art.

### SUMMARY OF THE INVENTION

It is the primary objective of the present invention to provide a method for using ¹²³I to radiolabel SnADAM and become an serotonin transporter radiotracer(¹²³I-ADAM).

According to the present invention, to use ¹²³I to radiolabel SnADAM and become an serotonin transporter radiotracer(¹²³I-ADAM). There is provided SnADAM solution via mixing SnADAM with ethanol. The SnADAM solution is shaken and further mixed with thin KI solution. The SnADAM solution is mixed with ¹²³I-NH₄I solution and H₂O₂ solution. The SnADAM-¹²³I-NH₄I—H₂O₂ mixture is kept still. Later, the SnADAM solution is mixed with NaHSO₃ solution, and the mixture is shaken and further mixed with buffer solution of saturated Na₂HPO₄. The SnADAM solution is filled in an Accubond C8 column. The Accubond C8 column is washed with sterile water for injection to isolate non-reacting ¹²³I ions. The Accubond C8 column is washed with ethanol, thus providing ¹²³I-ADAM. The ¹²³I-ADAM is blended in normal saline mixture. Millipore Millex GV is used to filter impurities and bacteria from the ¹²³I-ADAM solution.

There is disclosed a method for using ¹²³I to radiolabel SnADAM and become an serotonin transporter radiotracer(¹²³I-ADAM). At first, there is provided SnADAM solution via mixing SnADAM with ethanol. The SnADAM solution is shaken and further mixed with thin KI solution. The SnADAM solution is mixed with ¹²³I-NH₄I solution and H₂O₂ solution. The SnADAM-¹²³I-NH₄I—H₂O₂ mixture is kept still. Later, the SnADAM solution is mixed with NaHSO₃ solution, and the mixture is shaken and further mixed with buffer solution of saturated Na₂HPO₄. The SnADAM solution is filled in an Accubond C8 column. The Accubond C8 column is washed with sterile water for injection to isolate non-reacting ¹²³I ions. The Accubond C8 column is washed with ethanol, thus providing ¹²³I-ADAM. The ¹²³I-ADAM is blended in normal saline mixture. Millipore Millex GV is used to filter impurities and bacteria from the ¹²³I-ADAM solution.

Other objectives, advantages and features of the present invention will become apparent from the following description referring to the attached drawings.

### BRIEF DESCRIPTION OF THE DRAWINGS

The present invention will be described via the detailed illustration of the preferred embodiment referring to the drawings.

FIG. 1 shows a formula for producing ¹²³I-ADAM according to the preferred embodiment of the present invention.

FIG. 2 is a flow chart of a method for radio labeling ¹²³I-ADAM according to the preferred embodiment of the present invention.

FIG. 3 is a table of the throughput of ¹²³I-ADAM according to the preferred embodiment of the present invention.

FIG. 4 is a chart of the radiochemistry purity of ¹²³I-ADAM measured by HPLC according to the preferred embodiment of the present invention.

FIG. 5 is a table of the radiochemistry purity of ¹²³I-ADAM according to the preferred embodiment of the present invention.

FIG. 6 is a chart of the radiochemistry purity of ¹²³I-ADAM after 0 hour according to the preferred embodiment of the present invention.

FIG. 7 is a chart of the radiochemistry purity of ¹²³I-ADAM after 48 hours according to the preferred embodiment of the present invention.

FIG. 8 shows images taken of the brain of a SD rat by a microSPECT and microCT according to the preferred embodiment of the present invention.

FIG. 9 shows images taken of the brain of an ape by a microSPECT and microCT according to the preferred embodiment of the present invention.

### DETAILED DESCRIPTION OF EMBODIMENT

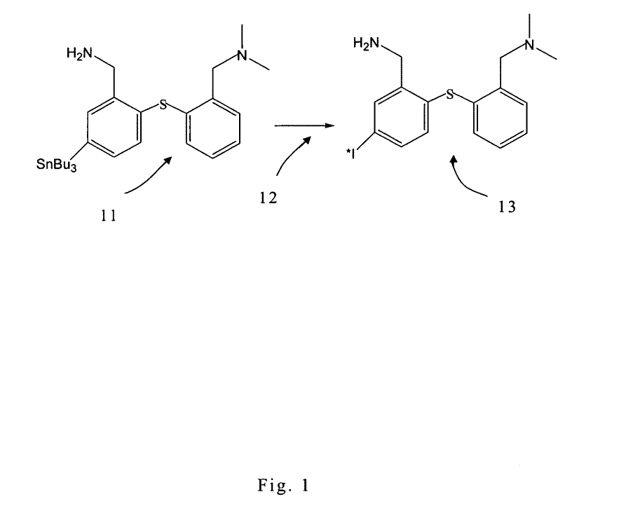

Referring to FIG. 1, according to the preferred embodiment of the present invention, SnADAM is provided as a precursor 11. The precursor 11 and ¹²³I-NH₄I are disposed in an acid environment. Solution 12 containing 5% of H₂O₂ is used for the oxidative de-tinning of the precursor 11. In the acid environment, tributyl tin is removed from the precursor 11. The H₂O₂ oxidizes the ¹²³I-NH₄I into iodine. Then, there is executed the covalent bonding of the iodine with the bonds from which the tributyl tin has been removed.

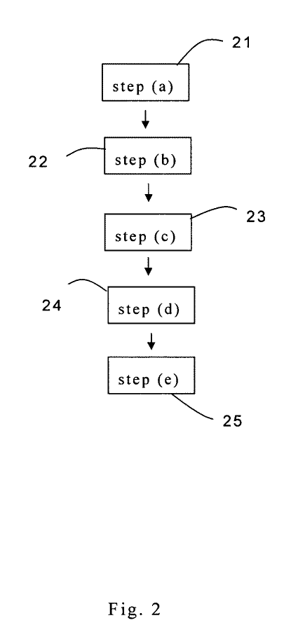

Referring to FIG. 2, at 21, 50 to 150 μg of SnADAM 11 is mixed with 50 μl of ethanol. The SnADAM-ethanol mixture is shaken for 30 seconds before it is further mixed with 4 μl of thin KI solution. Thus, SnADAM solution is provided.

At 22, there is provided 200 μl of ¹²³I-NH₄I solution. The radiochemistry activity of the ¹²³I-NH₄I solution is measured with a dose calibrator. The radiochemistry activity of the ¹²³I-NH₄I solution is about 200 mCi. The ¹²³I-NH₄I solution is mixed with the SnADAM solution before they are further mixed with 50 μl of H₂O₂ solution 12. The ¹²³I-NH₄I-SnADAM-H₂O₂ mixture is shaken before it is kept still for 5 minutes for further reaction to take place.

A 23, the SnADAM solution is mixed with 300 μl of solution containing 39% of NaHSO₃. The mixture is shaken before it is mixed with 2 ml of buffer solution of saturated Na₂HPO₄.

At 24, the solution of SnADAM is filled in an Accubond C8 column. Sterile water for injection is filled in the Accubond C8 column. The solution is pumped from the Accubond C8 column slowly. The Accubond C8 column is washed with the sterile water for injection for 10 times. Then, the Accubond C8 column is washed with 0.5 ml of solution containing 50% of ethanol. The cleaning liquid is poured from the Accubond C8 column. Then, the Accubond C8 column is washed with 900 μl of ethanol slowly, thus providing ¹²³I-ADAM 13.

At 25, the ¹²³I-ADAM 13 is mixed with 3.5 ml of normal saline mixture. Millipore Millex GV0.22 μm is used to filter impurities and bacteria from the solution of ¹²³I-ADAM. The product can be stored in glass bottles for use. The process takes about 40 minutes and is a rapid radio-labeling method.

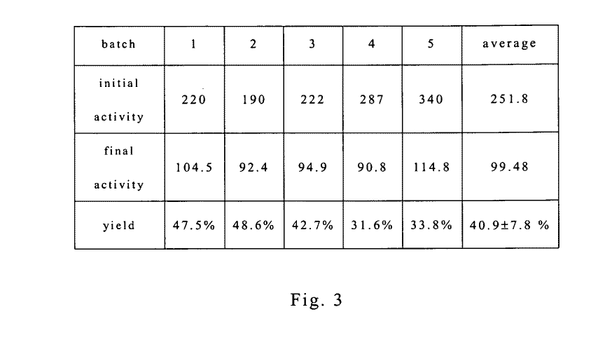

Referring to FIG. 3, the throughput of the ¹²³I-ADAM 13 is measured and results are shown. In five radio-labeling processes, the throughput of the ¹²³I-ADAM 13 has reached 40.9±7.8%.

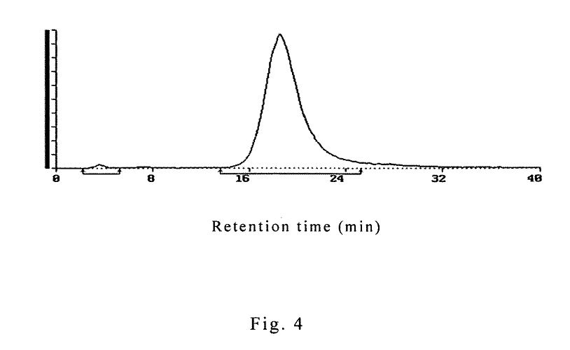

Referring to FIG. 4, an HPLC is used to measure the radiochemistry purity of the ¹²³I-ADAM 13. Reverse-phase high-performance liquid chromatography equipped with a radiodetector is used for the qualitative and quantitative assessment of the ¹²³I-ADAM 13. The retention time of the ¹²³I-ADAM 13 is 18 minutes.

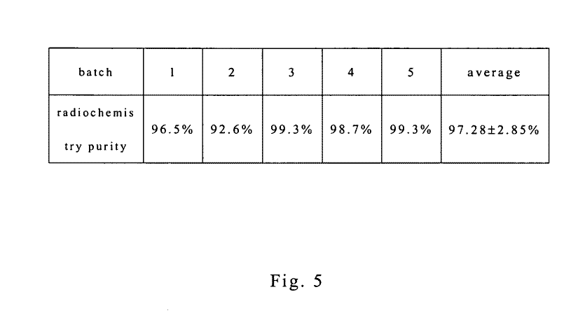

Referring to FIG. 5, the radiochemistry purity of the ¹²³I-ADAM is shown in a table. The HPLC determines that the radiochemistry purity of the ¹²³I-ADAM 13 is 97.28±2.85% (n=5).

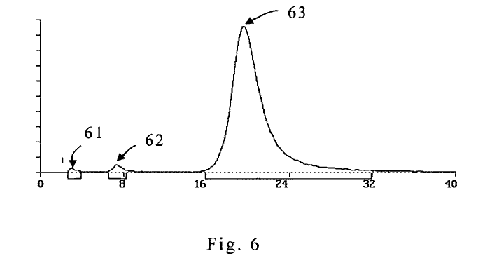

Referring to FIG. 6, the radiochemistry purity of the ¹²³I-ADAM after 0 hour is shown in a chart. At 61, the radiochemistry purity is 0.67%. At 62, the radiochemistry purity is 1.58%. At 63, the radiochemistry purity is 97.78%.

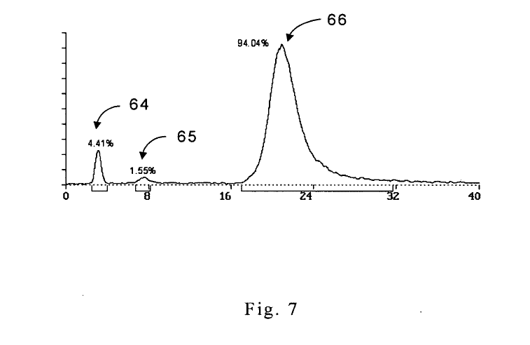

Referring to FIG. 7, the radiochemistry purity of the ¹²³I-ADAM after 48 hours is shown in a chart. At 64, the radiochemistry purity is 4.41%. At 65, the radiochemistry purity is 1.55%. At 66 the radiochemistry purity is 94.04%. After 48 hours, the radiochemistry purity is still higher than 90%.

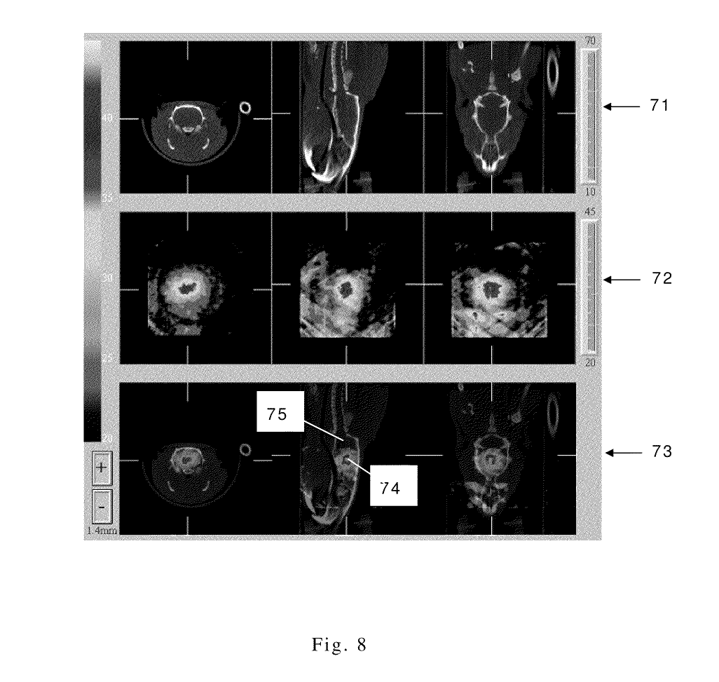

Referring to FIG. 8, a microCT image 71 and a microSPECT image 72 of the brain of a SD rat are shown. A fused image 73 of the microCT image and the microSPECT image 72 is also shown. The affinity of the ¹²³-ADAM 13 with the midbrain 74 of the SD rat is better than with the cerebellum 73. The concentration of the serotonin transporter in the midbrain is higher than in the cerebellum 73. The ¹²³I-ADAM 13 can effectively be used to detect the serotonin transporter.

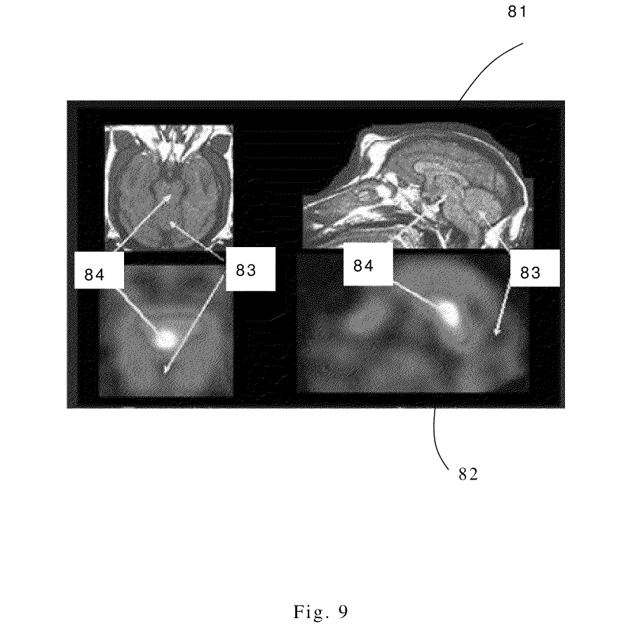

Referring to FIG. 9, the ¹²³I-ADAM is used to provide a SPECT image and a MRI image 82 of an ape. The affinity of the ¹²³I-ADAM 13 with the hypothalamus of the midbrain 84 of the ape is more affinitive than with the cerebellum 83. The concentration of the serotonin transporter in the hypothalamus is higher than in the cerebellum 83. The ¹²³I-ADAM 13 can effectively be used to detect the serotonin transporter.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

1. A method for using ¹²³I to radiolabel SnADAM become an serotonin transporter radiotracer(¹²³I-ADAM) comprising the steps of:
providing SnADAM solution via mixing SnADAM with ethanol, shaking the SnADAM-ethanol mixture, and further mixing it with thin KI solution;
mixing the SnADAM solution with ¹²³I-NH₄I solution and H₂O₂ solution and keeping the SnADAM-¹²³I-NH₄I—H₂O₂ mixture still;
mixing the SnADAM solution with NaHSO₃ solution, shaking the SnADAM-NaHSO₃ mixture, and further mixing it with buffer solution of saturated Na₂HPO₄;
filling the SnADAM solution in an Accubond C8 column, washing the Accubond C8 column with sterile water for injection to isolate non-reacting ¹²³I ions, and washing the Accubond C8 column with ethanol, thus providing ¹²³I-ADAM; and
blending the ¹²³I-ADAM in normal saline mixture, and using Millipore Millex GV to filter impurities and bacteria from the ¹²³I-ADAM solution.

2. The method according to claim 1, wherein there is provided 150 to 250 μl of ¹²³I-NH₄I solution.

3. The method according to claim 1, wherein the radiochemistry activity of the ¹²³I-NH₄I solution is 200 to 250 mCi.

4. The method according to claim 1, wherein the amount of the SnADAM is 50 to 150 μg.

5. The method according to claim 1, wherein the amount of the ethanol is 40 to 60 μl.

6. The method according to claim 1, wherein the amount of the thin KI solution is 1 to 6 μl.

7. The method according to claim 1, wherein the shaking lasts for 20 to 40 seconds.

8. The method according to claim 1, wherein the amount of the NaHSO₃ solution is 250 to 350 μl.

9. The method according to claim 1, wherein the mixture is kept still for 3 to 7 minutes.

10. The method according to claim 1, wherein the amount of the buffer solution of Na₂HPO₄ is 1 to 5 ml.

11. The method according to claim 1, wherein the amount of the normal saline mixture is 1 to 5 ml.

12. The method according to claim 1, wherein the Millipore Millex GV is 0.15 to 0.25 μm.
